(19) United States
(12) Patent Application Publication
(10) Pub. No.: US 2015/0320674 A1
(43) Pub. Date: Nov. 12, 2015

(54) Compositions Comprising Synthetic Waxes

(71) Applicants: David Gittleman, Croton on Hudson, NY (US); Deena Strauss Kornblau, Chappaqua, NY (US); Richard J. Czarnecki, Wayne, NJ (US)

(72) Inventors: David Gittleman, Croton on Hudson, NY (US); Deena Strauss Kornblau, Chappaqua, NY (US); Richard J. Czarnecki, Wayne, NJ (US)

(73) Assignee: MICRO POWDERS, INC., Tarrytown, NY (US)

(21) Appl. No.: 14/272,840

(22) Filed: May 8, 2014

(51) Int. Cl. A61K 8/92; A61Q 5/02; A61Q 19/10; A61K 8/02; A61Q 1/14

(52) U.S. Cl. CPC A61K 8/92; A61K 8/0241; A61Q 1/14; A61Q 19/10; A61Q 5/02; A61K 2800/41

(57) ABSTRACT

The present invention relates to certain novel synthetic wax powder and cosmetic composition comprising such synthetic wax powders. The present invention also relates to processes for manufacturing the wax powder having an irregular particle shape, methods of making cosmetic and personal care compositions and the use of such cosmetic compositions in topical applications.

### FIELD OF INVENTION

The present invention relates to certain novel synthetic wax powder compositions for personal care and cosmetic compositions. The present invention also relates to cosmetic compositions comprising at least one synthetic wax composition. The present invention further relates to processes for micronizing synthetic wax to create irregular shape particles, methods of making cosmetic and personal care compositions and the methods of using such cosmetic compositions in topical applications for improved exfoliating action.

The present invention also relates to cosmetic compositions, including fine particle cosmetic products, loose and packed cosmetic powders, foundations, eye shadows, scrub soaps, scrubs, exfoliating agents, creams, liquids, gels, lotions and compositions suitable for keratin materials made from synthetic wax. The compositions according to the invention are intended more particularly for the keratin materials of human beings, such as the skin (including the scalp), the nails and keratin fibers, and/or mucous membranes.

More particularly, the present invention relates to a cosmetic composition that can be used as a body scrub wherein the cosmetic composition comprises a synthetic wax having a substantially irregular particle shape.

### BACKGROUND

### Prior Art Cosmetics

Cosmetics have been around since Biblical times. Over the years cosmetics have been developed in many different formulations and in many different forms whether as a powder, liquid, gel or cream. These different formulations and forms have been used for a host of topical uses.

Cosmetic powders have been long known and commercially available in both loose and pressed powder form. Cosmetic powders have been used for a variety of topical application such to the face, lips, around the eyes, nails, scalp and other body locations. The uses of these cosmetic powders include reducing, inhibiting or preventing moisture loss from the skin for an extended period of time; absorbing excess sebum/oil from the skin to reduce shine and filling in blemishes.

Cleansing compositions have also been around for many years whether in powder, liquid or cream forms. Cleansing the skin is also very important especially for the care of the face. Cleansing must be as thorough as possible because greasy residues, such as excess sebum, dead skin cells, and remnants of cosmetic products used daily and make-up products accumulate in the folds of the skin and can block the pores of the skin and result in blemishes.

Various forms of cosmetic compositions for cleaning the skin have been known for years. For example, U.S. Pat. No. 3,645,904 discloses a skin cleaner having plastic synthetic resin material in a fine state of comminution so as to impart a scrubbing or mechanical detersive action thereto.

U.S. Pat. No. 6,103,249 discloses a cosmetic composition in the form of a soft paste that comprises at least one wax. The compositions of this patent can contain micronized polymers or plastics but there is no disclosure or teaching of compositions comprising micronized wax with an irregular non-uniform granular particle shape.

Similarly, U.S. Pat. No. 6,063,366 discloses a cosmetic composition comprising disintegrating granules that facilitate the circulation of the blood to improve a complexion. The disintegrating granules are composed of synthetic polymers such as polyethylene, polystyrene, polyester, polyvinyl chloride, polyamide, polypropylene, and nylon in a silicone solvent wherein the disintegrating granules in the cosmetic composition have a compression strength of 0.002 to 0.1 kgf/mm² and a granule size of 100 to 2,000 microns.

U.S. Pat. No. 6,309,655 discloses a cosmetic composition comprising self-heating, self-indicating disintegrating granules comprised of water-insoluble synthetic polymer and a colorant, which gives users indications of the length of time the composition has been applied and the degree of mixing when in use.

U.S. Pat. No. 7,195,770 discloses a body scrub wherein the cosmetic composition comprises crushed up basalt. Though basalt is a synthetic ingredient it is a mineral and not a synthetic wax.

U.S. Pat. No. 7,585,922 discloses polymer particles dispersions in a silicone medium and cosmetic compositions made therefrom. However, the object of this patent was to stabilize the polymer dispersion without the use of a stabilizing polymer. This was accomplished by modifying the physicochemical properties of the dispersion, and thus of the composition comprising it, by appropriately selecting the monomers and the silicone medium of which it is composed. The polymer particle dispersion of this patent comprised a copolymer that comprises at least one first block that is soluble in the silicone medium and at least one second block that is insoluble in the silicone medium.

U.S. Pat. No. 4,508,634 discloses an aqueous skin cleaner composition comprising propylene carbonate suitable for removing paint, grease, dirt and other foreign materials from the skin.

U.S. Publication No. 20120171142 discloses a composition containing an oil-dispersible micronized wax and at least one oil. The composition of this application has beneficial cosmetic properties including comfort upon application due to the preferred spherical nature of the oil-dispersible micronized wax. These compositions are used in lip gloss and other cosmetic products where a high shine is required without the drawback of feathering/migration.

As will be seen none of the prior art discloses the present invention. Further, until now, there has not been a cosmetic composition, including but not limited to a body scrub, comprising a synthetic wax powder having an irregular shape and that has the synthetic abrasive effectiveness and aesthetic desirability of the cosmetic composition of the present invention.

### Synthetic Waxes

Waxes, including synthetic waxes have been long employed in cosmetic compositions but never in micronized form and never as an abrasive agent.

Further, none of the prior art discloses synthetic waxes having irregular non-uniform granular shape in powder forms that can be incorporated into cosmetic body scrub compositions. Further, the prior art does not disclose micronizing synthetic waxes to create irregular non-uniform granular shape particles that have improved exfoliation properties as compared to mostly spherical or smooth particles. Likewise, none of the prior art discloses the use of micronized synthetic wax produced by the Fischer-Tropsch process in cosmetic compositions. More specifically, none of the prior art show use of micronized synthetic waxes having an irregular particle shape as mechanical abrasive agents for a body scrub composition. In addition, none of the prior art discloses or teach the use of micronized synthetic waxes in loose and packed powders. Further, the prior art does not disclose or teach replacing polymer or other plastic microbeads or particles with micronized waxes whether having an irregular or regular particle shape.

### SUMMARY OF THE INVENTION

Further areas of applicability of the present invention will become apparent from the detailed description provided hereinafter. It should be understood that the detailed description, while indicating the preferred embodiment of the invention, are intended for purposes of illustration only and are not intended to limit the scope of the invention.

The present inventors have also discovered, surprisingly, novel synthetic wax powder compositions in micronized form suitable for topical use, including use in skincare products, loose and packed cosmetic powders, cleansing scrubs and other cosmetic compositions.

It is another object of the invention to provide processes for caring for the keratin materials of human beings, comprising the application of a composition containing the polymer compositions defined above to the keratin materials.

It is yet another object of the present invention to provide a process for making cosmetic compositions using the micronized synthetic wax powder compositions.

The cosmetic powder composition of this invention may be any suitable cosmetic powder composition for application to any suitable area of skin, such as for application to the face, lips, nose, around the eye, the scalp, or any other suitable body area. The cosmetic powder compositions of the present invention may be free flowing, loose powder compositions or pressed powder compositions.

The present inventors have further discovered, surprisingly, novel synthetic wax powders wherein the wax particles have substantially irregular non-uniform granular shape, and cosmetic compositions suitable for topical applications. The term “particles” is synonymous with the term “microbeads” and refer generally to particles, grains, agglomerates, etc.

The present inventors have also discovered, surprisingly, powder compositions comprising at least one synthetic wax in micronized form with a substantially irregular non-uniform granular particle shape suitable for topical use, especially for use as a body scrub. As used herein, the expression “at least one” means one or more and thus includes individual components as well as mixtures/combinations.

The inventor has discovered that synthetic wax with substantially irregular non-uniform granular particle shape can be used as an exfoliate abrasive. Further, the inventor has discovered that these irregular non-uniform granular shaped synthetic wax particles can be used either to replace polymer or plastic particles or used in conjunction with polymer or plastic particles or microbeads to provide the mechanical abrasiveness necessary for a body scrub. This is becoming increasingly important as regulations are enacted to eliminate or limit the use of plastic particles and microbeads whether biodegradable or not, in cosmetics and personal care products. The irregular shaped wax particles of the present inventions have the requisite properties such as sufficient hardness and high melting temperatures necessary for particles used as mechanical scrubbing agents in cosmetic compositions especially body scrubs.

The present inventors have also discovered, surprisingly, powder compositions comprising at least one synthetic wax in micronized form with a substantially regular particle shape suitable for cosmetic powder compositions. These compositions of the present invention gives cosmetic compositions that are is easy to apply to the skin surface and provide good feel. As used herein, the expression “at least one” means one or more and thus includes individual components as well as mixtures/combinations.

It is another object of the present invention to provide a process for making cosmetic compositions using the micronized synthetic wax powder compositions.

The powder compositions of the present invention are designed to provide increased exfoliation as compared to powder compositions comprising mostly smooth or regular shape particles of wax or a polymer.

Further, the cosmetic compositions comprising the micronized synthetic wax comprise micronized synthetic wax particles of various sizes depending on the intended application. These compositions may also comprise other additives that are normally found in cosmetic compositions, again depending on the intended application.

These and other objects of the present invention will become more readily apparent from consideration of the following summary and detailed description. Further areas of applicability of the present invention will become apparent from the detailed description provided hereinafter. It should be understood that the detailed description, while indicating the preferred embodiment of the invention, are intended for purposes of illustration only and are not intended to limit the scope of the invention.

### DETAILED DESCRIPTION OF EXEMPLARY EXAMPLES

The present invention involves the development of novel synthetic wax powders in micronized form suitable for use in topical applications such as use cosmetic and personal care compositions. The novel synthetic wax powder compositions and scrub compositions of the present invention afford good cosmetic properties. In addition, the micronized synthetic wax particles have necessary properties, including hardness and melting temperature, that they can be used to replace polymer and plastic constituents of cosmetic compositions.

Synthetic waxes/synthetic paraffins of the present invention are waxes obtained by Fischer-Tropsch synthesis, i.e., Fischer-Tropsch waxes.

Synthetic waxes further include synthetic waxes that have been modified. “Modified” as used herein, means any chemical or physical alteration, including without limitation the addition of a pigment, dye or coloring agent to the synthetic wax. The wax of the present invention may also contain, if desired, suitable chemical and/or biological agents.

Synthetic waxes for use in cosmetic compositions of the present invention are commercially available and may be obtained from various chemical manufacturers in several forms. Synthetic wax of the present invention include but are not limited to synthetic wax sold by Micro Powders, Inc. under the names MicroEase 1145®, Synscrub 20PC®, Synscrub 50PC® and Microease 110XF®.

Additional synthetic waxes of the present invention include, but are not limited to, Sasolwax® sold by Sasol Wax GmbH. Examples of Sasolwax® of the invention include, but are not limited to, Sasolwax C80, Sasolwax H1, Sasolwax H105, Sasolwax SP 30 and Sasolwax SP 105. Another Fischer-Tropsch waxes of the present invention include Shell MDS waxes, including Shell GTL Sarawax SX50, Shell GTL Sarawax SX55R, Shell GTL Sarawax SX60S, Shell GTL Sarawax SX70, Shell GTL Sarawax SX100 and Shell GTL Sarawax SX105.

Other synthetic waxes, which can be used in the present invention, are commercially available in suitable form from one or more manufacturers. An Internet search can easily locate one or more suppliers of a synthetic wax raw material of the present invention.

An important feature of some of the synthetic waxes of the present invention is biodegradability. Biodegradability has become increasingly important, as regulations have been enacted to care for the environment. Biodegradable is broad and means that an object can be biologically broken down and should not be confused with the term compostable. A biodegradable synthetic wax of the present invention includes, but is not limited to, Sasolwax H105.

The terms “between . . . and . . . ” and “ranging from . . . to . . . ” mean that the limits are also included.

Synthetic waxes of the present invention preferably have melting points between 40° C. and 150° C. as measured by ASTM D 4591, and more preferably between 50° C. and 130° C. and even more preferably between 80° C. and 110° C. Melting points below 40° C. may result in a wax powder or cosmetic composition containing the wax powder in which some of the particles have lost their irregular non-uniform granular shape resulting in a less effective body scrub.

Other than in the operating examples, or where otherwise indicated, all numbers expressing quantities of ingredients, characteristics of ingredients and/or reaction conditions are to be understood as being modified in all instances by the term “about,” meaning within 10% to 15% of the indicated number.

A person of skill in the art will appreciate that additives or coatings can be added to the irregular wax particles that will assist in maintaining the irregular non-uniform granular particles shape of the wax powder even at higher temperatures or temperatures approaching the melting point of the particular wax. The only limitation for such additives and/or coatings is that a substantial percent of the wax particles making up the wax powder substantially retain an irregular shape.

Synthetic waxes of the present invention preferably have density at 25° C. between 0.70 (g/cc) and 1.50 (g/cc) as measured by ASTM D 792, and more preferably between 0.80 (g/cc) and 1.20 (g/cc) and even more preferably between 0.94 (g/cc) and 1.02 (g/cc).

A person of skill in the art will appreciate that additives or coatings can be added to the irregular wax particles that will increase the density or the wax powder. Such wax particles are within the invention with the only limitation that the wax particles making up the wax powder substantially retain an irregular shape.

### Micronizing Synthetic Waxes

The wax particles constituting one of the characteristics of the present invention are in a micronized form.

The synthetic waxes of the present invention that can be obtained commercially or produced using any known method for obtaining such wax cannot be directly used for the powder and cosmetic compositions of the present invention because they are in a solid form, flake form, or if obtained in a powder form have too large a particle size or do not have irregular non-uniform granular particle shape. Thus, the synthetic waxes have to be processed to obtain a micron sized particle size and an irregular non-uniform granular shape.

The micronizing process to employ depends largely on the use of the micronized synthetic wax and the desired particle shape.

### Micronizing to Create Irregular Shape Synthetic Wax Particles

The inventor has found processes for micronizing the synthetic waxes to give them irregular non-uniform granular shapes and make them suitable for use in powders and cosmetic composition of the present invention especially body scrub compositions. One or more processing methods may be required to obtain wax particles having the requisite size ranges and/or irregular non-uniform granular shape.

For use in the powder compositions and cosmetic compositions a synthetic wax must have the requisite size. The synthetic wax flakes or pellets that are not of the desired size can be processed to the required size according to methods generally known in the art.

One known method employs a grinding process. In this procedure, waxes present in the form of pastilles, prills, scales, flakes or coarse wax powders, are micronized by means of special grinding installations, for example jet or disk mills. Some of these mills are constructed so that only ground material having the desired particle size will leave the grinding chamber.

Furthermore, series-arranged grinding units are known which bring the waxes to the desired grain size in various stages. Finally, milling unit-screening unit combinations are utilized as well wherein the coarse product screened out after the first grinding pass is immediately returned to the grinding chamber and brought to the desired particle fineness in a second grinding pass. The grinding processes necessarily yield irregular particles having a large surface area.

One such grinding method is disk mill grinding. In disk mill grinding the feedstock is fed between opposing discs or plates that crush grind or chop the feedstock into smaller pieces. Because the larger pastilles, prills, scales, flakes or coarse wax powders are ground between the rotating discs, the resulting particles that are irregular shaped. Liquid nitrogen can be added to the system to control the temperature of the materials to ensure the material is friable enough to fracture and grind.

Air jet milling is another grinding method contemplated by the inventor to produce irregular-shaped wax powder particles. In air jet milling the feedstock is pushed at high speeds around the perimeter of the chamber by multiple jets of air or steam. As the particles are sped along the chamber the particles impact each other in high speed collisions that result in size reductions. No grinding media is involved. The interior of the impact chamber is designed to allow recirculation of over-sized particles, enhancing the incidence and the effect of these collisions. As particles are reduced in size and progressively lose mass, they migrate toward the central discharge port, making precise classification both automatic and precisely controllable. Particle size is adjusted primarily by a change in feed rate. When the rate is reduced, finer particles result because there is more energy available per particle to accelerate the particles and the entire fluid mass. Collisions become more violent and pressure gradients become larger. Particles produced by air jet milling necessarily have irregular shapes.

Other methods to obtain micronized wax particles with an irregular shape are within the invention with the only limitation that a substantial percent of the particles have the requisite size and irregular shape.

A person skilled in the art will be able to determine the necessary parameters for processing the synthetic wax into the necessary particle size of an irregular shape by methods generally known in the art, including by disk or air jet milling without undue experimentation. In addition, there are several commercial enterprises that have toll processing services that can be contracted to micronize the large synthetic wax to suitable sizes for the powder and cosmetic compositions of the present invention.

### Micronizing to Create Regular Shape Synthetic Wax Particles

Other compositions and uses of micronized synthetic wax powders and cosmetic compositions require susbstantially regular shape particles of micronized synthetic wax. Regular shapes particles include but are not limited to spherical or ellipsoidal or oval. The terms “spherical” or “ellipsoidal” or “oval” as used herein also mean that the solid wax particle has a uniform and substantially spherical or ellipsoidal or oval shape. The term “substantially” as used in the context of the shape of a spherical particle means that the particle is of substantially isotropic shape, i.e., it has a relatively regular morphology. It should be noted, however, that a line of symmetry is not required when the solid wax particle has a spherical shape.

Further, the micronized synthetic wax particles of the present invention may optionally have surface texturing, such as lines or indentations or protuberances that are relatively small in scale when compared to the overall size of the solid wax particle, and still be considered by those of skill in the art to be substantially spherical or ellipsoidal or oval.

The micronized synthetic wax particles according to the present disclosure may, in at least certain embodiments, be substantially homogeneous with respect to their shape and/or particle size distribution, although it is not required. The term “substantially” as used in this context means that about 50% or more of the solid wax particles in an aqueous dispersion of the present disclosure are of the same spherical, ellipsoidal, or oval shape and of the same particle size distribution.

A person skilled in the art will understand micronizing processes that will result in particles having a substantially regular shape.

One such method is a spraying process wherein a hot, low-viscosity wax melt is forced through a nozzle and atomized into very fine wax droplets as disclosed in U.S. Pat. No. 4,846,887 which is incorporated by reference in its entirety.

In addition, several companies offer micronized synthetic wax particles having a substantially regular shape and a person of skill in the art would readily obtain such wax particles.

### Synthetic Wax Powder Compositions of the Invention

Once the synthetic wax is micronized it can be incorporated into powder compositions suitable for use in cosmetic compositions. The powder compositions of the present invention are preferably ultimately formulated into powder and skincare compositions, but can also be formulated into a wide variety of product types, including rinses, hand and body lotions, mousses, gels, lotions, tonics, sprays, shampoos, conditioners, facial moisturizers, sunscreens, anti-acne preparations, topical analgesics, mascaras, and the like.

The powder compositions of the present invention are preferably ultimately formulated into body scrubs.

The micronized powder compositions of the present invention can be in an amount from 0.5% to 100% by weight micronized synthetic wax powder. One or more additional components can be added with the only caveat being that they are cosmetically compatible with the synthetic wax powder. These additives can be added in a net amount of from 0.1% to 99.5% and more preferably in the amount of 10% to 50%. Preferred additives include waxes, polymers and binders.

When an additive is to be added to the micronized wax powder it is preferable to make a homogeneous mixture or blend. When one or more other waxes are to be used in the composition with micronized wax it is preferable to melt the wax or waxes and the micronized wax together then micronizing the resulting mixture to provide a homogeneous blend.

The micronized powder compositions of the present invention can have substantially an irregular non-uniform granular shape. Thus, it is contemplated as within the present invention a powder composition comprising particles having a regular particle shape. Wax powders having between 1.0% and 100% irregular non-uniform granular shape particles as compared to the total are within the present invention. More preferably the percent or irregular shape particles should be greater than 1.0% and even more preferably greater than 3.0%. Below 0.5%, body scrub begins to lose its mechanical exfoliate properties.

Wax powders having a maximum particles size between 0.1 microns and 1410 microns (14 mesh) are within the present invention. More preferably the maximum particle size of the wax particles should be between 74 microns (200 mesh) and 1410 microns (14 mesh) and even more preferably between 150 microns (100 mesh) and 1410 microns (14 mesh). The particle size ranges include all specific values and sub-ranges therebetween. The particle size may be measured with the machine “Microtrac X100 & SRA 150” from Leeds-Northrup as per ASTM D4464 standard or by standard sieve analysis as described in ASTM E-11. Above or below these sizes the body scrub begins to lose its effectiveness as a body scrub.

However, it is contemplated as within the invention a wax powder compositions employing two or more different powders whether from the same source or different sources, having different mean particle sizes, having different particle shapes and having different percentage of particle shapes.

Numerous powder combinations or particle size and percent of irregular percentage of particles are also within the invention. A person of skill in the art will appreciate that the ultimate use of the powder compositions may dictate the right combination of particle size and percent of irregular shape particles. By way of example only, a low percent of irregular shape wax powder particles and/or a powder with a smaller particle size may be more suitable for body scrubs targeted to sensitive parts of the body such as the face. Likewise, a powder having a high percent of irregularly shaped particles' or larger particle size may be more suitable for a body scrub for coarser parts of the body such as the feet or elbows.

In an embodiment of the invention, if other wax(es) are present in the composition, the micronized synthetic wax constitutes the main wax of the composition (that is, the composition contains more micronized wax than all other waxes combined on a weight basis, containing, for example, 51%, 60%, 70%, 80%, 90%, 95% micronized synthetic wax as compared to all other waxes present on a weight basis).

One or more additional components can be added to the wax powder compositions with the only caveat being that they are cosmetically compatible with the synthetic wax powder. These additives can be added in a net amount of from 0.1% to 99.5% and more preferably in the amount of 10% to 50%. Preferred additives include other waxes, polymers and binders.

### Body Scrub Compositions of the Invention

The micronized powder compositions of the present invention can be formulated into products for topical administration, including body scrubs. The term “body scrub” as used herein refers to an abrasive cleaning composition used to remove dead skin from the human body such as, for example, from the hands, feet, elbows, and knees.

The body scrub compositions according to the invention can contain a large percentage of the synthetic wax powder. The body scrub compositions according to the invention preferably contain synthetic wax powder in an amount of from 1% to 20%, more preferably from 3% to 7.5%, and even more preferably 3% to 5% by weight.

The body scrub compositions according to the invention have wax particle size (or number-average particle size) which can range in particular from about 200 mesh to about 14 mesh, preferably from 120 mesh to 20 mesh, and more preferably from 100 mesh to 20 mesh. The particle size ranges include all specific values and sub-ranges therebetween. The particle size is determined using the ASTM E11 standard with an Alpine Sieve. The particle size chosen will depend on the intended application and the abrasive effect desired. Particle sizes above the listed ranges will produce too much abrasive effect and lead to irritation of the skin of the user. Particle sizes below the listed range value will be too small to provide the necessary abrasive effect required for the user. Thus, the applicant has found the listed ranges to be ideally suited for cosmetic scrub compositions of the present invention.

### Powder Cosmetic Compositions of the Invention

The micronized powder compositions of the present invention can be formulated into products for topical administration, including into powder cosmetics.

The powder cosmetic compositions of the present invention preferably contain micronized synthetic wax powder in an amount of from 1% to 30%, more preferably from 5 to 10% by weight.

The powder compositions according to the invention can have particle size (or number-average particle size) which can range in particular from about 0.1 to about 44 microns, preferably with a mean particle size of 5 to 20 microns with a maximum particle size of 44 microns, and more preferably with a mean particle size of 8 to 12 microns with a maximum particle size of 31.11 microns. The particle size ranges include all specific values and sub-ranges therebetween.

Particle size above 44 microns the powder will not have a good sensation or texture and the user will “feel” the powder particles upon application which will lead to discomfort.

### Other Materials and Additives

Other materials may be present in the compositions of the present invention. In the selection of ingredients in the compositions of the present invention, it is contemplated that materials will be utilized that are compatible, both chemically and physically, with other the synthetic waxes. Such materials include, but are not limited to, cosmetically acceptable diluents or carriers, binders, pigments, pharmacological agents, surfactants, excipients and fillers.

Suitable pigments for use herein can be inorganic and/or organic. Also included within the term pigment are materials having a low color or luster such as matte finishing agents, and also light scattering agents. Examples of suitable pigments are iron oxides, acylglutamate iron oxides, ultramarine blue, D&C dyes, carmine, and mixtures thereof.

Also suitable for use herein especially from the viewpoint of moisturization, skin feel, skin appearance and emulsion compatibility are treated pigments. Pigments can be treated with compounds such as amino acids (e.g., lysine), silicones, lauroyl, collagen, polyethylene, lecithin and ester oils. The more preferred pigments are the silicone (polysiloxane) treated pigments.

In addition, the cosmetic compositions of the invention may also comprise any additive usually used in cosmetics and personal care compositions according to the present invention, such as antioxidants, fillers, preserving agents, fragrances, neutralizing agents, thickeners, cosmetic or dermatological active agents such as, for example, emollients, moisturizers, vitamins and sunscreens, and mixtures thereof. Preferred ranges of additives is from about 0.5% to about 20% by weight, preferably from about 1% to about 10%, and more preferably from about 2% to about 5% by weight of the total composition.

The body scrub compositions of the present invention can also include any cosmetically acceptable agent that is compatible, both chemically and physically, with one or more synthetic wax. Such materials include, but are not limited to, cosmetically acceptable diluents or carriers, binders, pigments, pharmacological agents, surfactants, excipients and fillers. Preferred ranges for the cosmetically acceptable agent is from about 5% to about 90% by weight, preferably from about 30% to about 80%, and more preferably from about 60% to about 80% by weight of the total composition.

Examples of possible additives can be found in U.S. Pat. Nos. 7,632,873; 7,670,999; 7,410,636 and 7,351,418 all of which are incorporated by reference in their entirety herein. Further examples of suitable additional components can be found in the other references which have been incorporated by reference in this application. Still further examples of such additional ingredients may be found in the International Cosmetic Ingredient Dictionary and Handbook (15^{th} ed. 2014).

Surfactants whether foaming or non-foaming may also be employed in the compositions of the present invention. The foaming surfactants used may be nonionic, anionic, amphoteric or zwitterionic surfactants. A person skilled in the art will be able to choose a surfactant to meet the need of the composition without undue experimentation. Further, examples of foaming surfactants can be found in U.S. Pat. No. 7,655,702 which is specifically incorporated by reference in its entirety herein. Preferred ranges for the surfactants is from about 1% to about 60% by weight, preferably from about 10% to about 50%, and more preferably from about 20% to about 40% by weight of the total composition.

In addition, the cosmetic compositions according to the present invention, can include ingredients commonly used in the classical external skin care compositions, skin cleansing compositions, cosmetic compositions and massaging compositions, for example, oily substances, anti-melanogenic agents, sebum secretion inhibitors, blood circulation-facilitating agents, softeners, surfactants, keratin protecting agents, thickeners, antiseptics, pH adjusters, perfume bases, colorants, medicinally-effective agents, solvents, cosmetic or dermatological active agents such as, for example, emollients, moisturizers, vitamins and sunscreens, and mixtures, may be suitably incorporated in addition to the above-described components so far as no detrimental influence is thereby imposed on the effects of the present invention. Preferred ranges for theses additives is from about 1% to about 60% by weight, preferably from about 10% to about 50%, and more preferably from about 20% to about 40% by weight of the total composition.

Other optional ingredients which can be included in the compositions of the invention include preservatives in amounts generally about 1% or less by weight. Suitable preservatives include methylparaben, propylparaben, imidazolidimyl urea, phenoxyethanol, and mixtures thereof. The compositions may also contain fragrances, sunscreens and chelating agents.

Needless to say, a person skilled in the art will take care to select the optional additional additives and/or the amount thereof such that the advantageous properties of the composition according to the invention are not, or are not substantially, adversely affected by the addition envisaged.

### Methods of Use of the Cosmetic Compositions Comprising Synthetic Wax

The cosmetic compositions including body scrub compositions of the present invention comprising synthetic wax powder having an irregular non-uniform granular particle shape can be used to cleanse the skin and other surface of an animal and more preferably a human. The synthetic wax particles having a substantial irregular non-uniform granular shape provide an improved scrubbing action to assist in cleaning the skin and keratin surfaces as compared to compositions comprising spherical or regular shaped particles.

On preferred method of cleaning the skin comprises applying an effective amount of the cosmetic compositions of the present invention to the surface to be cleaned and using gentle pressure preferably in a circular motion to contact the cosmetic compositions to the total area to be cleaned. This action should be done for a sufficient time. The cosmetic composition then is rinsed off preferably with water.

### Manufacture of the Cosmetic Compositions

The composition according to the invention may be manufactured by the known processes generally used in cosmetics or dermatology. For example, the compositions may be prepared by mixing the materials by any conventional means or any means known to one of ordinary skill in the art including, but not limited to, mechanical mixers.

The following examples will more fully illustrate the embodiments of this invention. It will be understood that the following examples are illustrative and not meant to limit the invention in any way. All parts, percentages and proportions referred to herein and in the appended claims are by weight unless otherwise illustrated.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

### Example 1

### Foaming Body Scrub

A foaming body scrub comprising synthetic wax powder with a maximum particle size of 840 microns and a maximum mesh size of 20 and the ingredients listed below was made according to the following:

| Product Name | INCI Name | % W/W | Supplier |
|---|---|---|---|
| Phase A | | | |
| Deionized water | Water | 51.20 | N/A |
| Carbopol Ultrez 20 | Carbomer | 0.60 | The Lubrizol Corporation |
| | | | (Wickliffe, Ohio) |
| Phase B | | | |
| Propylene Glycol | Propylene Glycol | 3.50 | Many |
| Germaben II | Propylene Glycol, | 1.00 | International Specialty Products |
| | Diazolidinyl Urea, | | (Wayne, New Jersey) |
| | Methylparaben, | | |
| | Propylparaben | | |
| Phase C | | | |
| Protachem ES-2 | Sodium Laureth Sulfate | 21.00 | Protameen Chemicals Inc. (Totowa, |
| | | | NJ) |
| Proteric CAB-LC | Cocamidopropyl | 7.00 | Protameen Chemicals Inc. (Totowa, |
| | Betaine | | NJ) |
| Protasorb L-20 | Polysorbate 20 | 1.50 | Protameen Chemicals Inc. (Totowa, |
| | | | NJ) |
| Protamide CME | Cocamide MEA | 3.50 | Protameen Chemicals Inc. (Totowa, |
| | | | NJ) |
| Phase D | | | |
| Deionized Water | Water | 3.00 | N/A |
| Disodium EDTA | Disodium EDTA | 0.05 | Many |
| Phase E | | | |
| Triethanolamine 99% | Triethanolamine | 0.65 | Many |
| Phase F | | | |
| Synscrub 20PC | Synthetic Wax | 7.00 | Micro Powders Inc. |

First, the Phase A solution is created by gentling sprinkling in the Carbopol Ultrez 20 on the surface of warm deionized water in a large vessel. (Heat the water to approximately 40° C.). Wait until the polymer has completely wetted out and then begin gentle mixing of the solution using any conventional mixing technique or apparatus known by those skilled in the art for 10 minutes. While the Phase A solution mixes, the Phase B solution is created. In a separate vessel, the Phase B materials are combined. Once the Phase A solution has mixed for ten minutes the Phase B solution is added to the Phase A solution. The combined solution (Phase AB) is then mixed until a homogeneous solution is created.

Once the Phase AB solution is homogeneous, the ingredients listed for Phase C are added individually and in the order listed above by a slow continuous mixing with a kitchen aid blender. It is important to make sure each ingredient is completely dispersed before beginning to add the next ingredient listed for Phase C.

In a separate vessel the Phase D solution is created by adding the listed ingredient by a slow continuous mixing process until completely dispersed. Phase D is then added to main batch which contains Phase A, B and C. The Phase E ingredient is then added to the batch and the pH is adjusted to 6.0-6.5 with TEA 99% component. The combined solution is then mixed until uniform. Then the Phase F is added slowly and again the solution is mixed until uniform.

The synthetic wax used in the above formulation has a maximum particle size of 840 microns and a mesh size of 20 as measured using the ASTM E11 test method and an Alpine Sieve.

The resulting foaming body scrub is environmentally friendly and has the same high performance as body scrubs made with synthetic non-biodegradable polymer particles. The resulting scrub composition has many and diverse cosmetic applications, including use as soap scrubs, foot scrubs, exfoliating face scrub, body scrubs, creams, liquids, gels and lotions.

### Example 2

### Foaming Body Scrub

A foaming body scrub comprising synthetic wax with a maximum particle size of 297 microns and a maximum mesh size of 50 and the ingredients listed below was made according to the following:

| Product Name | INCI Name | % W/W | Supplier |
|---|---|---|---|
| Phase A | | | |
| Deionized water | Water | 51.20 | N/A |
| Carbopol Ultrez 20 | Carbomer | 0.60 | The Lubrizol Corporation |
| | | | (Wickliffe, Ohio) |
| Phase B | | | |
| Propylene Glycol | Propylene Glycol | 3.50 | Many |
| Germaben II | Propylene Glycol, | 1.00 | International Specialty Products |
| | Diazolidinyl Urea, | | (Wayne, New Jersey) |
| | Methylparaben, | | |
| | Propylparaben | | |
| Phase C | | | |
| Protachem ES-2 | Sodium Laureth Sulfate | 21.00 | Protameen Chemicals Inc. (Totowa, |
| | | | NJ) |
| Proteric CAB-LC | Cocamidopropyl | 7.00 | Protameen Chemicals Inc. (Totowa, |
| | Betaine | | NJ) |
| Protasorb L-20 | Polysorbate 20 | 1.50 | Protameen Chemicals Inc. (Totowa, |
| | | | NJ) |
| Protamide CME | Cocamide MEA | 3.50 | Protameen Chemicals Inc. (Totowa, |
| | | | NJ) |
| Phase D | | | |
| Deionized Water | Water | 3.00 | N/A |
| Disodium EDTA | Disodium EDTA | 0.05 | Many |
| Phase E | | | |
| Triethanolamine 99% | Triethanolamine | 0.65 | Many |
| Phase F | | | |
| Synscrub 50PC | Synthetic Wax | 7.00 | Micro Powders Inc. |

First, the Phase A solution is created by gentling sprinkling in the Carbopol Ultrez 20 on the surface of warm deionized water in a large vessel. (Heat the water to approximately 40° C.). Wait until the polymer has completely wetted out and then begin gentle mixing of the solution using any conventional mixing technique or apparatus known by those skilled in the art for 10 minutes. While the Phase A solution mixes, the Phase B solution is created. In a separate vessel, the Phase B materials are combined. Once the Phase A solution has mixed for ten minutes the Phase B solution is added to the Phase A solution. The combined solution (Phase AB) is then mixed until a homogeneous solution is created.

Once the Phase AB solution is homogeneous, the ingredients listed for Phase C are added individually and in the order listed above 1 by a slow continuous mixing with a CBM Mixer (6 qt, tumble speed 35, impeller speed 3500) for 1 minute until evenly dispersed. until evenly dispersed.

It is important to make sure each ingredient is completely dispersed before beginning to add the next ingredient listed for Phase C.

In a separate vessel the Phase D solution is created by adding the listed ingredient by a slow continuous mixing process until completely dispersed. Phase D is then added to main batch which contains Phase A, B and C. The Phase E ingredient is then added to the batch and the pH is adjusted to 6.0-6.5 with TEA 99% component. The combined solution is then mixed until uniform. Then the Phase F is added slowly and again the solution is mixed until uniform.

The synthetic wax used in the above formulation has a maximum particle size of 297 and a mesh size of 50 mesh as measured using the ASTM E11 test method on an Alpine Sieve.

The resulting foaming body scrub is environmentally friendly and has the same high performance as body scrubs made with synthetic polymer particles. The resulting scrub composition has many and diverse cosmetic applications, including use as soap scrubs, foot scrubs, exfoliating face scrub, body scrubs, creams, liquids, gels and lotions.

An advantage of the compositions of the present invention is that the compositions are composed of biodegradable synthetic wax as opposed to synthetic polymers. This is particularly desirable since the cosmetic compositions that can be created with the compositions of the present invention are for use on the human body. The compositions of the present invention and the cosmetic compositions created therefrom are ideally suited for use at home as well as at a salon or a spa.

The subsequent use of the wax powder and wax powder compositions of the invention depends on the desired aim and on the active agents which may be present. It can be used in particular to care for, cleanse, remove make-up from, make up and/or treat the human skin, scalp and/or mucous membranes.

Thus, another subject of the present invention is a cosmetic process to care for, cleanse, remove make-up from, make up and/or treat the skin, mucous membranes and/or the scalp, comprising the application of a powder as defined above to the skin, mucous membranes and/or the scalp.

Another subject of the invention is the use of the wax powder as defined above for the preparation of a composition intended to care for, cleanse, remove make-up from, make up and/or treat the skin, the scalp and/or mucous membranes.

### Example 3

### Pressed Powder

A pressed powder compositions comprising synthetic wax with a mean particle size of 8.0 to 12.0 microns and the ingredients listed below was made according to the following:

| | | % | |
|---|---|---|---|
| Product Name | INCI Name | W/W | Supplier |
| Phase A | | | |
| Talc Micro Ace P-2 | Talc | 40.00 | Presperse LLC (Somerset, NJ) |
| Unipure Yellow LC182 | Iron Oxide | 1.75 | Sensient Cosmetic Techs. (South |
| Oxide | | | Plainfield, NJ) |
| Unipure Brown LC881 | Iron Oxide | 1.20 | Sensient Cosmetic Techs. (South |
| Oxide | | | Plainfield, NJ) |
| Unipure Black LC989 | Iron Oxide | 0.10 | Sensient Cosmetic Techs. (South |
| Oxide | | | Plainfield, NJ) |
| Potassium Sorbate | Potassium Sorbate | 0.20 | Lotioncrafter LLC. (Olga, WA) |
| Tetrasodium EDTA | Tetrasodium EDTA | 0.05 | Lotioncrafter LLC. (Olga, WA) |
| Protachem LL | Lauroyl Lysine | 2.00 | Protameen Chemicals Inc. (Totowa, NJ) |
| Talc Micro Ace P-2 | Talc | 36.40 | Presperse LLC (Somerset, NJ) |
| Phase B | | | |
| Sericite SL-012 | Mica (and) Methicone | 9.00 | Presperse LLC (Somerset, NJ) |
| Microease 110XF | Synthetic Wax | 4.90 | Micro Powders Inc. |
| Phase C | | | |
| Protachem ISP | Isotearyl Palmitate | 3.55 | Protameen Chemicals Inc. (Totowa, NJ) |
| Vitamin E-Acetate | DL-alpha-tocopheryl | 0.10 | BASF Corp. (Florham Park, NJ) |
| | acetate | | |
| Barguard CP | Caprylyl Glycol | 0.75 | Paradigm Research Science |
| | Phenoxyethanol, | | |
| | Hexylene Glycol | | |

The ingredients listed for Phase A were combined in the order listed above. The mixture was passed through a micropulverizer twice such as a Hammermill with a 0.020 or 0.035 herringbone screen or a 4″ Jetmill @ 40 g/min (2.4 kg/hr). The powder phase was drawn down to check for streaking. If needed, the powder composition is re-pulverized to ensure uniformity of phase/color. The Phase B materials are then added to the Phase A composition and the combined mixture is blended well with a CBM Mixer (6 qt, tumble speed 35, impeller speed 3500) for 1 minute until evenly dispersed.

The micronized synthetic wax particles used in the above formulation has a mean particle size of 8.0 to 12.0 microns as measured using a Microtrac device and the ASTM D4464 test method.

The Phase C materials are pre-mixed separately and then added to the Phase AB composition and the resulting composition is blended well with a CBM Mixer (6 qt, tumble speed 35, impeller speed 3500) for 1 minute until evenly dispersed.

The resulting powder compositions have a good slip resistance and texture. The resulting powder compositions have many and diverse cosmetic applications, including use as foundations, eye shadow, blushes, skincare preparations, mascaras, creams, gels, and lotions.

### Example 4

### Loose Face Powder

A loose face powder compositions comprising synthetic wax with a mean particle size of 8.0 to 12.0 microns and the ingredients listed below was made according to the following:

| Product Name | INCI Name | % W/W | Supplier |
|---|---|---|---|
| Phase A | | | |
| Talc Micro Ace P-2 | Talc | 44.04 | Presperse LLC (Somerset, NJ) |
| Unipure Red LC381 | Iron Oxide | 0.73 | Sensient Cosmetic Techs. (South |
| Oxide | | | Plainfield, NJ) |
| Unipure Yellow LC182 | Iron Oxide | 1.23 | Sensient Cosmetic Techs. (South |
| Oxide | | | Plainfield, NJ) |
| Unipure Black LC989 | Iron Oxide | 0.65 | Sensient Cosmetic Techs. (South |
| Oxide | | | Plainfield, NJ) |
| Phase B | | | |
| Sericite SL-012 | Mica (and) Methicone | 5.00 | Presperse LLC (Somerset, NJ) |
| Microease 110XF | Synthetic Wax | 4.90 | Micro Powders Inc. |
| Talc Micro Ace P-2 | Talc | 30.00 | Presperse LLC (Somerset, NJ) |
| Phase C | | | |
| Dermol 258 | C12-C15 Alkyl | 1.75 | ALZO Int'l (Sayreville, NJ) |
| | Octanoate | | |
| BHT | Butylated | 0.05 | Many |
| | hydroxytoluene | | |
| Barguard CP | Caprylyl Glycol, | 0.75 | Paradigm Research Science |
| | Phenoxyethanol, | | |
| | Hexylene Glycol | | |
| Phase D | | | |
| PresPearl Elegant Gold | Mica (and) Titanium | 4.00 | Presperse LLC (Somerset, NJ) |
| | Oxide (and) Iron | | |
| | Oxide | | |
| PresPearl Ruby Red | Mica (and) Iron Oxide | 1.30 | Presperse LLC (Somerset, NJ) |
| PresPearl | Mica (and) Titanium | 0.50 | Presperse LLC (Somerset, NJ) |
| | Dioxide (and) Iron | | |
| | Oxide | | |

The ingredients listed for Phase A were combined in the order listed above. The mixture was passed through a micropulverizer twice such as a Hammermill with a 0.020 or 0.035 herringbone screen or a 4″ Jetmill @ 40 g/min (2.4 kg/hr). The powder phase was drawn down to check for streaking. If needed, the powder composition is re-pulverized to ensure uniformity of phase/color. The Phase B materials are then added to the Phase A composition and the combined mixture is blended well with a CBM Mixer (6 qt, tumble speed 35, impeller speed 3500) for 1 minute until evenly dispersed until evenly dispersed.

The synthetic wax used in the above formulation has a mean particle size of 8.0 to 12.0 microns as measured using a Microtrac device and the ASTM D4464 test method.

The Phase C materials are pre-mixed. Slight heat may be required to ensure that the BHT is dissolved in the phase. However, in no event, should the Phase C composition be heated above 40 degrees Celsius. The Phase C composition is then added to the Phase AB composition and the resulting composition is blended well until the materials are evenly dispersed. The Phase D materials are then added to the Phase ABC composition and the composition is blended well in a CBM Mixer (6 qt, tumble speed 35, impeller speed 3500) for 1 minute until evenly dispersed until the materials are evenly dispersed.

The resulting powder compositions have a good slip resistance and texture. The resulting powder compositions have many and diverse cosmetic applications, including use as foundations, eye shadow, blushes, skincare preparations, mascaras, creams, gels, and lotions.

### Example 5

### Loose Mineral Powder

A loose mineral powder comprising synthetic wax with a mean particle size of 8.0 to 12.0 microns and the ingredients listed below was made according to the following:

| Product Name | INCI Name | % W/W | Supplier |
|---|---|---|---|
| Phase A | | | |
| Sericite PHN | Mica | 62.94 | Presperse LLC (Somerset, NJ) |
| Satin B-UVS | Bismuth Oxychloride | 15.00 | Presperse LLC (Somerset, NJ) |
| Unipure Yellow LC182 | Iron Oxide | 1.73 | Sensient Cosmetic Techs. (South |
| Oxide | | | Plainfield, NJ) |
| Unipure Red LC381 | Iron Oxide | 1.28 | Sensient Cosmetic Techs. (South |
| Oxide | | | Plainfield, NJ) |
| Unipure Black LC989 | Iron Oxide | 0.60 | Sensient Cosmetic Techs. (South |
| Oxide | | | Plainfield, NJ) |
| Zinc Stearate | Zinc Stearate | 2.00 | Sun Ace Kakoh Ltd (Singapore) |
| Phase B | | | |
| Sericite SL-012 | Mica (and) Methicone | 5.00 | Presperse LLC (Somerset, NJ) |
| Microease 110XF | Synthetic Wax | 4.90 | Micro Powders Inc. |
| Phase C | | | |
| Ceraphyl 368 | Ethylhexyl Palmitate | 0.50 | International Specialty Products |
| | | | (Wayne, New Jersey) |
| Dermol 258 | C12-C15 Alkyl | 0.35 | ALZO Int'l (Sayreville, NJ) |
| | Octanoate | | |
| Barguard CP | Caprylyl Glycol, | 0.75 | Paradigm Research Science |
| | Phenoxyethanol, | | |
| | Hexylene Glycol | | |

The ingredients listed for Phase A were combined in the order listed above. The mixture was passed through a micropulverizer twice such as a Hammermill with a 0.020 or 0.035 herringbone screen or a 4″ Jetmill @ 40 g/min (2.4 kg/hr). The powder phase was drawn down to check for streaking. If needed, the powder composition is re-pulverized to ensure uniformity of phase/color. The Phase B materials are then added to the Phase A composition and the combined mixture is blended well with a CBM Mixer (6 qt, tumble speed 35, impeller speed 3500) for 1 minute until evenly dispersed. until evenly dispersed.

The synthetic wax used in the above formulation has a mean particle size of 8.0 to 12.0 microns as measured using a Microtrac device and the ASTM D4464 test method.

The Phase C materials are pre-mixed separately and then added to the Phase AB composition and the resulting composition is blended well with a CBM Mixer (6 qt, tumble speed 35, impeller speed 3500) for 1 minute until evenly dispersed.

The resulting powder compositions have a good slip resistance and texture. The resulting powder compositions have many and diverse cosmetic applications, including use as foundations, eye shadow, blushes, skincare preparations, mascaras, creams, gels, and lotions.

### Example 6

### Pressed Powder

A pressed powder compositions comprising synthetic wax with a mean particle size of 8.0 to 12.0 microns and the ingredients listed below was made according to the following:

| | | % | |
|---|---|---|---|
| Product Name | INCI Name | W/W | Supplier |
| Phase A | | | |
| Talc Micro Ace P-2 | Talc | 40.00 | Presperse LLC (Somerset, NJ) |
| Unipure Yellow LC182 | Iron Oxide | 1.75 | Sensient Cosmetic Techs. (South |
| Oxide | | | Plainfield, NJ) |
| Unipure Brown LC881 | Iron Oxide | 1.20 | Sensient Cosmetic Techs. (South |
| Oxide | | | Plainfield, NJ) |
| Unipure Black LC989 | Iron Oxide | 0.10 | Sensient Cosmetic Techs. (South |
| Oxide | | | Plainfield, NJ) |
| Potassium Sorbate | Potassium Sorbate | 0.20 | Lotioncrafter LLC. (Olga, WA) |
| Tetrasodium EDTA | Tetrasodium EDTA | 0.05 | Lotioncrafter LLC. (Olga, WA) |
| Protachem LL | Lauroyl Lysine | 2.00 | Protameen Chemicals Inc. (Totowa, NJ) |
| Talc Micro Ace P-2 | Talc | 36.40 | Presperse LLC (Somerset, NJ) |
| Phase B | | | |
| Sericite SL-012 | Mica (and) Methicone | 9.00 | Presperse LLC (Somerset, NJ) |
| Microease 110S | Synthetic Wax | 4.90 | Micro Powders Inc. |
| Phase C | | | |
| Protachem ISP | Isotearyl Palmitate | 3.55 | Protameen Chemicals Inc. (Totowa, NJ) |
| Vitamin E-Acetate | DL-alpha-tocopheryl | 0.10 | BASF Corp. (Florham Park, NJ) |
| | acetate | | |
| Barguard CP | Caprylyl Glycol | 0.75 | Paradigm Research Science |
| | Phenoxyethanol, | | |
| | Hexylene Glycol | | |

The ingredients listed for Phase A were combined in the order listed above. The mixture was passed through a micropulverizer twice such as a Hammermill with a 0.020 or 0.035 herringbone screen or a 4″ Jetmill @ 40 g/min (2.4 kg/hr). The powder phase was drawn down to check for streaking. If needed, the powder composition is re-pulverized to ensure uniformity of phase/color. The Phase B materials are then added to the Phase A composition and the combined mixture is blended well with a CBM Mixer (6 qt, tumble speed 35, impeller speed 3500) for 1 minute until evenly dispersed.

The micronized synthetic wax particles used in the above formulation has a mean particle size of 8.0 to 12.0 microns as measured using a Microtrac device and the ASTM D4464 test method.

The Phase C materials are pre-mixed separately and then added to the Phase AB composition and the resulting composition is blended well with a CBM Mixer (6 qt, tumble speed 35, impeller speed 3500) for 1 minute until evenly dispersed.

The resulting powder compositions have a good slip resistance and texture. The resulting powder compositions have many and diverse cosmetic applications, including use as foundations, eye shadow, blushes, skincare preparations, mascaras, creams, gels, and lotions.

### Example 7

### Loose Face Powder

A loose face powder compositions comprising synthetic wax with a mean particle size of 8.0 to 12.0 microns and the ingredients listed below was made according to the following:

| Product Name | INCI Name | % W/W | Supplier |
|---|---|---|---|
| Phase A | | | |
| Talc Micro Ace P-2 | Talc | 44.04 | Presperse LLC (Somerset, NJ) |
| Unipure Red LC381 | Iron Oxide | 0.73 | Sensient Cosmetic Techs. (South |
| Oxide | | | Plainfield, NJ) |
| Unipure Yellow LC182 | Iron Oxide | 1.23 | Sensient Cosmetic Techs. (South |
| Oxide | | | Plainfield, NJ) |
| Unipure Black LC989 | Iron Oxide | 0.65 | Sensient Cosmetic Techs. (South |
| Oxide | | | Plainfield, NJ) |
| Phase B | | | |
| Sericite SL-012 | Mica (and) Methicone | 5.00 | Presperse LLC (Somerset, NJ) |
| Microease 110S | Synthetic Wax | 4.90 | Micro Powders Inc. |
| Talc Micro Ace P-2 | Talc | 30.00 | Presperse LLC (Somerset, NJ) |
| Phase C | | | |
| Dermol 258 | C12-C15 Alkyl | 1.75 | ALZO Int'l (Sayreville, NJ) |
| | Octanoate | | |
| BHT | Butylated | 0.05 | Many |
| | hydroxytoluene | | |
| Barguard CP | Caprylyl Glycol, | 0.75 | Paradigm Research Science |
| | Phenoxyethanol, | | |
| | Hexylene Glycol | | |
| Phase D | | | |
| PresPearl Elegant Gold | Mica (and) Titanium | 4.00 | Presperse LLC (Somerset, NJ) |
| | Oxide (and) Iron | | |
| | Oxide | | |
| PresPearl Ruby Red | Mica (and) Iron Oxide | 1.30 | Presperse LLC (Somerset, NJ) |
| PresPearl | Mica (and) Titanium | 0.50 | Presperse LLC (Somerset, NJ) |
| | Dioxide (and) Iron | | |
| | Oxide | | |

The ingredients listed for Phase A were combined in the order listed above. The mixture was passed through a micropulverizer twice such as a Hammermill with a 0.020 or 0.035 herringbone screen or a 4″ Jetmill @ 40 g/min (2.4 kg/hr). The powder phase was drawn down to check for streaking. If needed, the powder composition is re-pulverized to ensure uniformity of phase/color. The Phase B materials are then added to the Phase A composition and the combined mixture is blended well with a CBM Mixer (6 qt, tumble speed 35, impeller speed 3500) for 1 minute until evenly dispersed until evenly dispersed.

The synthetic wax used in the above formulation has a mean particle size of 8.0 to 12.0 microns as measured using a Microtrac device and the ASTM D4464 test method.

The Phase C materials are pre-mixed. Slight heat may be required to ensure that the BHT is dissolved in the phase. However, in no event, should the Phase C composition be heated above 40 degrees Celsius. The Phase C composition is then added to the Phase AB composition and the resulting composition is blended well until the materials are evenly dispersed. The Phase D materials are then added to the Phase ABC composition and the composition is blended well in a CBM Mixer (6 qt, tumble speed 35, impeller speed 3500) for 1 minute until evenly dispersed until the materials are evenly dispersed.

The resulting powder compositions have a good slip resistance and texture. The resulting powder compositions have many and diverse cosmetic applications, including use as foundations, eye shadow, blushes, skincare preparations, mascaras, creams, gels, and lotions.

### Example 8

### Loose Mineral Powder

A loose mineral powder comprising synthetic wax with a mean particle size of 8.0 to 12.0 microns and the ingredients listed below was made according to the following:

| Product Name | INCI Name | % W/W | Supplier |
|---|---|---|---|
| Phase A | | | |
| Sericite PHN | Mica | 62.94 | Presperse LLC (Somerset, NJ) |
| Satin B-UVS | Bismuth Oxychloride | 15.00 | Presperse LLC (Somerset, NJ) |
| Unipure Yellow LC182 | Iron Oxide | 1.73 | Sensient Cosmetic Techs. (South |
| Oxide | | | Plainfield, NJ) |
| Unipure Red LC381 | Iron Oxide | 1.28 | Sensient Cosmetic Techs. (South |
| Oxide | | | Plainfield, NJ) |
| Unipure Black LC989 | Iron Oxide | 0.60 | Sensient Cosmetic Techs. (South |
| Oxide | | | Plainfield, NJ) |
| Zinc Stearate | Zinc Stearate | 2.00 | Sun Ace Kakoh Ltd (Singapore) |
| Phase B | | | |
| Sericite SL-012 | Mica (and) Methicone | 5.00 | Presperse LLC (Somerset, NJ) |
| Microease 110S | Synthetic Wax | 4.90 | Micro Powders Inc. |
| Phase C | | | |
| Ceraphyl 368 | Ethylhexyl Palmitate | 0.50 | International Specialty Products |
| | | | (Wayne, New Jersey) |
| Dermol 258 | C12-C15 Alkyl | 0.35 | ALZO Int'l (Sayreville, NJ) |
| | Octanoate | | |
| Barguard CP | Caprylyl Glycol, | 0.75 | Paradigm Research Science |
| | Phenoxyethanol, | | |
| | Hexylene Glycol | | |

The ingredients listed for Phase A were combined in the order listed above. The mixture was passed through a micropulverizer twice such as a Hammermill with a 0.020 or 0.035 herringbone screen or a 4″ Jetmill @ 40 g/min (2.4 kg/hr). The powder phase was drawn down to check for streaking. If needed, the powder composition is re-pulverized to ensure uniformity of phase/color. The Phase B materials are then added to the Phase A composition and the combined mixture is blended well with a CBM Mixer (6 qt, tumble speed 35, impeller speed 3500) for 1 minute until evenly dispersed. until evenly dispersed.

The synthetic wax used in the above formulation has a mean particle size of 8.0 to 12.0 microns as measured using a Microtrac device and the ASTM D4464 test method.

The Phase C materials are pre-mixed separately and then added to the Phase AB composition and the resulting composition is blended well with a CBM Mixer (6 qt, tumble speed 35, impeller speed 3500) for 1 minute until evenly dispersed.

The resulting powder compositions have a good slip resistance and texture. The resulting powder compositions have many and diverse cosmetic applications, including use as foundations, eye shadow, blushes, skincare preparations, mascaras, creams, gels, and lotions.

It will therefore be readily understood by those persons skilled in the art that the present invention is susceptible of broad utility and application. Many embodiments and adaptations of the present invention other than those herein described, as well as many variations, modifications and equivalent arrangements, will be apparent from or reasonably suggested by the present invention and the foregoing description thereof, without departing from the substance or scope of the present invention. Accordingly, while the present invention has been described herein in detail in relation to its preferred embodiment, it is to be understood that this disclosure is only illustrative and exemplary of the present invention and is made merely for purposes of providing a full and enabling disclosure of the invention. The foregoing disclosure is not intended or to be construed to limit the present invention or otherwise to exclude any such other embodiments, adaptations, variations, modifications and equivalent arrangements.

Obviously additional modifications and variations of the present invention are possible in light of the above teachings. It is therefore to be understood that within the scope of the appended claims, the invention may be practiced otherwise than as specifically described herein.

1. A wax powder composition suitable for use in a cosmetic composition, comprising a synthetic wax.

2. A wax powder composition suitable for use in a cosmetic composition, comprising a synthetic wax wherein the synthetic wax has an irregular non-uniform granular particle shape.

3. The wax powder composition of claim 1 wherein the synthetic wax is present in an amount from 0.5% to 100% % by weight.

4. The composition according to claim 1 wherein the synthetic wax has a mean particle sizes from about 150 to 200 microns.

5. The composition according to claim 1 further comprising an additive in the amount from about 0.1% to 99.5% by weight.

6. The composition of claim 1 wherein the synthetic wax is a Fischer-Tropsch wax.

7. The composition of claim 1 wherein the synthetic wax is biodegradable.

8. A cosmetic composition comprising:
(a) from about 1% to about 30% by weight of wax particles having an irregular non-uniform irregular shape; and
(b) a cosmetically acceptable agent; wherein said composition is in the form of a cosmetic scrub.

9. The composition of claim 5 wherein the synthetic wax particles are present in an amount from about 1% to about 10% by weight.

10. The composition of claim 15 wherein the synthetic wax have particle size ranges from about 14 mesh to about 100 mesh.

11. The composition according to claim 8 further comprising a surfactant present in amount from about 1% to about 60% by weight.

12. The composition according to claim 8 wherein the cosmetically acceptable agent is present in an amount from about 5% to about 90% by weight.

13. The composition of claim 8 wherein the synthetic wax is a Fischer-Tropsch wax.

14. The composition of claim 8 wherein the synthetic wax is biodegradable.

15. A method of cleansing and/or removing makeup from the skin, the eyes, the scalp and/or the hair comprising: contacting a composition of claim 8 to the skin, the eyes, the scalp and/or the hair thereby cleansing and/or removing make-up from the skin, the eyes, the scalp and/or the hair.

16. The method of claim 15 further comprising rinsing said skin with water or wiping said skin with a substrate.

17. A composition for topical application comprising:
(a) from about 1% to about 30% by weight of a micronized synthetic wax; and
(b) one or more cosmetic powder base components selected from the group consisting of pigments, matte finishing agents, light scattering agents, fillers and binders, and mixture thereof; wherein said composition is in the form of a powder.

18. The composition of claim 17 wherein the micronized synthetic wax has a particle size from about 0.1 microns to about 44 microns.

19. The composition of claim 17 wherein the micronized synthetic wax has a substantially regular particle shape.

20. The composition of claim 17 wherein the micronized synthetic wax is present in an amount from about 5% to about 10% by weight.

21. The composition according to claim 17 wherein the binder is a wax present in amount from about 1% to about 30% by weight.

22. The composition of claim 17 further comprising an additive present in an amount from about 0.5% to about 20% by weight.

23. The composition of claim 17 wherein the synthetic wax is a Fischer-Tropsch wax.

24. The composition of claim 17 wherein the synthetic wax is biodegradable.

25. A method of preparing a cosmetic composition comprising:
(a) preparing a synthetic wax powder by micronizing the wax; and
(b) admixing about 3 to 20 weight percent, based upon the total weight of the composition, of the aggregate with a cosmetically acceptable carrier.
